(19) United States
(12) Patent Application Publication
(10) Pub. No.: US 2026/0130951 A1
(43) Pub. Date: May 14, 2026

(54) GLYCYRRHIZIN-GLYCOL CHITOSAN CONJUGATE-COATED IRON OXIDE NANOPARTICLES AND USE THEREOF

(71) Applicant: Industry-University Cooperation Foundation Hanyang University, Seoul (KR)

(72) Inventors: Dong Yun LEE, Seoul (KR); Su Bin JANG, Seoul (KR); Sangjoon LEE, Seoul (KR)

(73) Assignee: Industry-University Cooperation Foundation Hanyang University, Seoul (KR)

(21) Appl. No.: 19/435,214

(22) Filed: Dec. 29, 2025

Related U.S. Application Data
(62) Division of application No. 17/042,400, now abandoned.

(30) Foreign Application Priority Data
Mar. 28, 2018 (KR) 10-2018-0036157

(51) Int. Cl. A61K 35/39; A61K 9/51; A61K 47/54; A61K 47/69; A61P 3/10; B82Y 5/00

(52) U.S. Cl. CPC A61K 35/39; A61K 9/5161; A61K 47/554; A61K 47/6929; A61P 3/10; B82Y 5/00

(57) ABSTRACT

The present invention relates to glycyrrhizin-glycol chitosan conjugate-coated nanoparticles, islet cells, prepared using same, for transplantation, and an MRI imaging composition comprising same. If transplanted, the islet cells comprising the nanoparticles can suppress a post-transplantation immune response. The present invention can provide islet cells for transplantation that can be transplanted to a certain region by magnetic force induction and can be tracked by MRI.

### CROSS REFERENCE TO RELATED APPLICATIONS

This application is a Divisional Application of U.S. application No. Ser. No. 17/042,400 filed Sep. 28, 2020, which is a National Stage of International Application No. PCT/KR2019/003522 filed Mar. 26, 2019, claiming priority based on Korean Patent Application No. 10-2018-0036157 filed Mar. 28, 2018.

### TECHNICAL FIELD

The present invention relates to glycyrrhizin-glycol chitosan conjugate-coated nanoparticles and an islet cell composition for transplantation using the same.

### BACKGROUND ART

The onset of type 1 diabetes reduces the number of beta cells exhibiting normal functions in the pancreas, and results in inadequate insulin secretion. Accordingly, blood glucose is not normally regulated, so that hyperglycemia occurs, which causes various complications.

As a method of treating such type 1 diabetes, a treatment method of inducing a temporary blood glucose regulation effect by artificially injecting insulin with an insulin syringe has been most widely used to date. In order to overcome the problem of temporary blood glucose regulation of the insulin injection method, transplantation methods of xenogeneic islet cells have been extensively studied. Although most of the cells are clinically transplanted into a liver site, there is a disadvantage in that an instant blood mediated inflammatory reaction (IBMIR) is induced because the cells are injected through the portal vein. Further, since a very small amount of islet cells are injected into a very wide liver site, there is also a problem in that it is difficult to track the islet cells. For these reasons, the marginal pancreatic islet mass required to regulate blood glucose control is extremely high. This is also a factor that makes the islet cell transplant surgery itself difficult due to the limitation of organ donors. Therefore, there is a need for a technique capable of clinically successfully engrafting islet cells in a liver tissue site of a patient with diabetes and continuously observing the viability and functionality thereof.

### DISCLOSURE

### Technical Problem

An object of the present invention is to provide a composition for transplanting islet cells, including biocompatible polymer-glycyrrhizin conjugate-coated nanoparticles, and islet cells treated with the composition, in order to overcome the loss of xenogeneic islet cells due to blood flow, which is the biggest problem of islet cell transplantation, and the autoimmune response which occurs after transplantation.

Another object of the present invention is to provide a pharmaceutical composition for alleviating, preventing, or treating an islet cell-deficiency disease, the pharmaceutical composition including the islet cells for transplantation.

Still another object of the present invention is to provide an MRI imaging composition including biocompatible polymer-glycyrrhizin conjugate-coated nanoparticles.

### Technical Solution

To achieve the objects, an aspect of the present invention provides a composition for transplanting islet cells, including nanoparticles coated with a biocompatible polymer-glycyrrhizin conjugate, in which the biocompatible polymer-glycyrrhizin conjugate is linked by a covalent bond.

The main cause of an autoimmune response occurring after xenogeneic islet cell transplantation is a high mobility group protein B1 (HMGB1) protein released from the nucleus of islet cells. The HMGB1 protein is produced in the nucleus and released out of the cell when physical stress is induced from the outside of the cell on the cell. The released HMGB1 protein binds to receptors (TLR, RAGE) of external immune cells to activate the immune cells and secrete cytokines from the immune cells. The secreted cytokines again apply external stimuli to the pancreatic islet cells, which causes the pancreatic islet cells to repeat a vicious cycle of internally expressing and releasing more HMGB1 protein, resulting in the loss of large amounts of transplanted pancreatic islet cells.

Accordingly, in order to reduce the level of autoimmune response that occurs during xenogeneic islet cell transplantation, the HMGB1 protein produced in the cell nucleus needs to be suppressed from being released out of the cell. For this purpose, the present inventors synthesized a biocompatible polymer backbone-glycyrrhizin conjugate by allowing glycyrrhizin capable of capturing an HMGB1 protein to bind to a biocompatible polymer backbone by a covalent bond.

As used herein, the term “glycyrrhizin” is an ingredient extracted from licorice and directly binds to the A box domain of the HMGB1 protein to inhibit activity and extracellular release. However, glycyrrhizin has a very short half-life in the body, and for example, when about 5 mg/kg of glycyrrhizin is intravenously injected, glycyrrhizin does not remain in the body for more than about 15 minutes, so that there is a limitation in the commercialization of glycyrrhizin as a pharmaceutical preparation.

In the present invention, the effect thereof could be enhanced by allowing such glycyrrhizin to bind to a biocompatible polymer backbone to increase the half-life in the body and increase the amount of glycyrrhizin absorbed into the cell.

In an exemplary embodiment of the present invention, glycyrrhizin of the present invention may be modified within a range of not losing inherent properties, and may be, for example, in an oxidized form. For the glycyrrhizin in the oxidized form, as illustrated in FIG. 1, a bond between the 2nd and 3rd carbons of the terminal glucuronic acid ring is opened by oxidation to form an aldehyde substituent. The aldehyde group thus formed may allow glycyrrhizin to form a bond with the biocompatible polymer backbone.

As used herein, the term “biocompatible polymer backbone” refers to a polymer having tissue compatibility and blood compatibility, which do not destroy a biological tissue or coagulate blood by bringing the biocompatible polymer backbone into contact with the tissue or blood, and refers to a polymer which serves as a backbone capable of forming bonds with a number of glycyrrhizin molecules.

In an exemplary embodiment of the present invention, the biocompatible polymer backbone of the present invention includes a functional group capable of forming a chemical bond with an aldehyde group. As described above, glycyrrhizin in the oxidized form includes an aldehyde group, and it can be used without limitation as long as a biocompatible polymer includes a substituent capable of forming a chemical bond with an aldehyde group of glycyrrhizin. For example, the functional group included in the biocompatible polymer backbone of the present invention is an amine group, a carboxyl group, a thiol group, or a hydroxide group. Preferably, a biocompatible polymer backbone including an amine group may be used.

In an exemplary embodiment of the present invention, the biocompatible polymer backbone of the present invention is selected from glycol chitosan, poly-L-lysine, poly(4-vinylpyridine/divinylbenzene), chitin, poly(butadiene/acrylonitrile) amine terminated, polyethyleneimine, polyaniline, poly(ethylene glycol)bis(2-aminoethyl), poly(N-vinylpyrrolidone), poly(vinylamine)hydrochloride, poly(2-vinylpyridine), poly(2-vinylpyridine N-oxide), poly-ε-Cbz-L-lysine, poly(2-dimethylaminoethyl methacrylate), poly(allylamine), poly(allylamine hydrochloride), poly(N-methylvinylamine), poly(diallyldimethylammonium chloride), poly(N-vinylpyrrolidone), chitosan, or poly(4-aminostyrene). Preferably, glycol chitosan may be used as the biocompatible polymer backbone.

The glycol chitosan is a material having bioaffinity and biodegradability, and is used in various fields such as tissue engineering or drug delivery. However, in the case of high molecular weight glycol chitosan, it was difficult to use the glycol chitosan as a pharmaceutical preparation due to a problem of toxicity due to its positive charge.

In an exemplary embodiment of the present invention, the biocompatible polymer backbone and glycyrrhizin are linked to each other by a covalent bond, and the covalent bond may be selected from the group consisting of an amide bond, a carbonyl bond, an ester bond, a thioester bond, and a sulfonamide bond. More preferably, the covalent bond may be an amide bond formed by reacting a carbonyl group of glycyrrhizin oxidized by treatment with sodium periodate or the like with an amine group of a biocompatible polymer backbone.

One of the most used methods for uptake of external materials into cells is naturally occurring endocytosis. However, the method using endocytosis is time-consuming, has difficulties in uptake of a sufficient amount of a material, and does not allow external materials to be uniformly absorbed into each cell, so that the method still has a limitation in use as a pharmaceutical preparation. Therefore, the present inventors used a method of coating nanoparticles with a glycyrrhizin-biocompatible polymer backbone conjugate in order to uniformly uptake a large amount of glycyrrhizin-biocompatible polymer backbone conjugate into islet cells and facilitate movement to a target site of the islet cell which have uptaken the conjugate.

As used herein, the term “nanoparticle” refers to a structure or material having a nanometer (nm) size. The nanometer size is a size of a micrometer (10⁻⁶) reduced by 1/1,000, and when the size of a material is decreased to the nanometer size, various and unique physical, chemical, mechanical, and electronic properties are exhibited.

In the present invention, an average size of the nanoparticles may be generally in a range of about 1 nm to about 500 nm, for example, about 1 nm to about 50 nm, about 50 nm to about 100 nm, about 100 nm to about 250 nm, and about 250 nm to about 500 nm.

According to an exemplary embodiment of the present invention, the nanoparticles of the present invention may be prepared in a size of 1 to 100 mm, preferably 1 to 50 mm, and more preferably 1 to 20 mm. When the size of nanoparticles is 500 nm or more, the sedimentation rate is increased, which causes inconvenience in use, so that a dispersant such as glucose, sucrose, glycerol, polyethylene amine, and betaine needs to be used to prevent sedimentation.

In the present invention, the nanoparticles may be organic or inorganic nanoparticles, and may be preferably magnetic nanoparticles.

As used herein, the term “magnetic” refers to the magnetic properties exhibited by a material. All materials interact with a magnetic field to generate an attractive force or a repulsive force. That is, when a magnetic field is applied to a substance, the substance is magnetized, and depending on the manner in which the object is magnetized, the object is classified into a ferromagnetic substance, a paramagnetic substance, a diamagnetic substance, a ferrimagnetic substance, and the like.

As used herein, the term “magnetic nanoparticles” refers to nanometer-sized structures or materials exhibiting magnetic properties. The magnetic nanoparticles may be prepared by solution synthesis, co-precipitation, sol-gel methods, high energy milling, hydrothermal synthesis, microemulsion synthesis, synthesis by thermal decomposition, or sonochemical synthesis, but the preparation method is not limited thereto.

In the present invention, the magnetic nanoparticles may be selected from the group consisting of iron (Fe), cobalt (Co), nickel (Ni), manganese (Mn), gadolinium (Gd), oxides thereof, or alloys thereof, but are not limited thereto.

According to an exemplary embodiment of the present invention, the magnetic nanoparticles are magnetic nanoparticles formed of iron or iron oxide.

In the present invention, as a general method for coating nanoparticles with a glycyrrhizin-biocompatible polymer backbone conjugate, it is possible to use i) a method of attaching a functional group present in the biocompatible polymer to nanoparticles, ii) a method of grafting a biocompatible polymer onto the surface of pre-synthesized nanoparticle or attaching a biocompatible polymer to the surface of pre-synthesized nanoparticle by click chemistry, iii) a method of using a block polymer including a block having a functional group capable of binding to nanoparticles as a biocompatible polymer, iv) a method of using a biocompatible polymer having a functional group (grafting group) capable of wrapping nanoparticles, v) a coating method by an ionic bond between the biocompatible polymer and the nanoparticles, or vi) a coating method using a hydrophobic-hydrophobic binding action with the surface of a hydrophobic nanoparticle after preparing a micelle form using an amphipathic polymer having hydrophilic and hydrophobic functional groups as a biocompatible polymer, but the coating method is not limited thereto.

According to an exemplary embodiment of the present invention, the biocompatible polymer includes a functional group capable of binding to nanoparticles, and the functional group may be an amine group, a carboxyl group, or a hydroxide group, and preferably a hydroxide group. Therefore, the thermal decomposition technique used in the preparation of magnetic nanoparticles may be used for nanoparticle coating. Specifically, by reacting iron oxide nanoparticles with a glycyrrhizin-glycol chitosan conjugate at high temperature, the conjugate may be wrapped on the surface of the iron oxide nanoparticle by an —OH functional group present in a polysaccharide of glycol chitosan, This method has an advantage in that the surface of the nanoparticle is stably coated with the conjugate by a large amount of —OH functional groups, but when the reaction is performed at high temperature for an excessively long time, a disadvantage in that the polysaccharide itself is decomposed may occur.

Another aspect of the present invention provides a pharmaceutical composition for alleviating, preventing, or treating an islet cell-deficiency disease, the pharmaceutical composition including, as an active ingredient, islet cells including the composition for transplanting islet cells into the cell.

Islet cells including a composition for transplanting islet cells including GC-SPIO in the cells have increased cell viability during in vivo transplantation, and thus may be used for all diseases which may occur due to islet cells-deficiency. The on/off method described in Example 3 may be used as a method for uptake of a composition for islet cell transplantation into the islet cells.

In an exemplary embodiment of the present invention, the islet cell-deficiency disease of the present invention is a disease selected from the group consisting of type 1 diabetes, type 2 diabetes, and a diabetic chronic kidney disease. The type 1 diabetes, the type 2 diabetes and the diabetic chronic kidney disease are all diseases treatable by islet cell transplantation.

In an exemplary embodiment of the present invention, the islet cell-deficiency disease of the present invention is type 1 diabetes. Since type 1 diabetes occurs when beta cells of the pancreatic islets which secrete insulin are destroyed and the insulin secretory function is lost, islet cell transplantation may be a good treatment method for type 1 diabetes.

A pharmaceutically acceptable carrier contained in the pharmaceutical composition of the present invention is typically used during formulation, and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, and the like, but is not limited thereto. The pharmaceutical composition of the present invention may additionally contain a lubricant, a wetting agent, a sweetening agent, a flavoring agent, an emulsifier, a suspending agent, a preservative, and the like, in addition to the aforementioned ingredients. Suitable pharmaceutically acceptable carriers and formulations are described in detail in Remington's Pharmaceutical Sciences (19th ed., 1995).

A suitable dose of the pharmaceutical composition of the present invention may vary depending on factors, such as formulation method, administration method, age, body weight, sex or disease condition of the patient, diet, administration time, administration route, excretion rate and response sensitivity. Meanwhile, the dose of the pharmaceutical composition of the present invention is preferably 0.001 to 1000 mg/kg (body weight) daily.

The pharmaceutical composition of the present invention may be prepared in the form of a unit-dose or by being contained in a multi-dose container by being formulated using a pharmaceutically acceptable carrier and/or excipient according to a method that can be readily implemented by a person with ordinary skill in the art to which the present invention pertains. In this case, a dosage form may also be in the form of a solution in an oil or aqueous medium, a suspension or in the form of an emulsion, an extract, a powder, a granule, a tablet or a capsule, and the pharmaceutical composition of the present invention may additionally include a dispersant or a stabilizer.

Still another aspect of the present invention provides a method for preparing islet cells for transplantation, the method including the following steps:
(a) bringing the composition for transplanting islet cells of item 1 into contact with islet cells isolated from a donor;
(b) applying a magnetic force to the islet cells for 0.1 to 5 minutes; and
(c) mixing the resulting product of (b) for 0.1 to 5 minutes.

In an exemplary embodiment of the present invention, the bringing of the composition for transplanting islet cells into contact with the islet cells isolated from the donor may include a method of culturing islet cells, and then treating the culture medium with the composition for transplanting islet cells.

In an exemplary embodiment of the present invention, Step (b) may be performed by a method of generating a magnetic force by bringing a magnet into contact with one surface of an islet cell culture plate, and as the used magnet, a neodymium magnet, an electromagnet, and the like may be used without limitation.

In an exemplary embodiment of the present invention, Step (c) is a step of mixing the resulting product such that the composition for transplanting islet cells is uniformly present in the islet cell culture medium, and the mixing method may include pipetting or stirring. The pipetting may be performed for 0.1 minute to 5 minutes, preferably for 0.5 minute to 3 minutes.

In an exemplary embodiment of the present invention, the method for preparing islet cells for transplantation may further include (d) allowing the islet cells to stand for 0.1 minute to 5 minutes after Step (c), and may further include allowing the islet cells to stand preferably for 0.5 minute to 3 minutes after Step (c).

In an exemplary embodiment of the present invention, for the method for preparing islet cells, Steps (b) to (d) may be repeated 1 time to 20 times, and preferably, Steps (b) to (d) may be repeated 5 times to 12 times.

As a result of studying a method capable of efficiently introducing GC-SPIO into islet cells, the present inventors confirmed that when the steps of applying a magnetic force, pipetting, and allowing the islet cells to stand were repeated after bringing GC-SPIO into contact with the islet cells, the uptake efficiency of GC-SPIO into islet cells was remarkably increased.

In the present invention, the method for uptake of GC-SPIO in islet cells, including the steps of applying a magnetic force, pipetting and allowing the islet cells to stand, is named an “on-off system”. The GC-SPIO absorbed into the islet cells captures the HMGB1 protein moving to the cytoplasm from the cell nucleus, and then the HMGB1 protein is decomposed by cellular activity. As a result, the autoimmune response is reduced because immune cell activation does not occur, so that the islet cells for transplantation prepared by the above method have increased viability, and as a result, long-term insulin secretion enables blood glucose to be regulated.

According to an exemplary embodiment of the present invention, the islet cells for transplantation prepared by the above method are responsive to a magnetic force by GC-SPIO included in the cells. Specifically, the largest amount of xenogeneic islet cells is transplanted into Mediate 1 of the liver lobes at the time of xenogeneic islet cell transplantation, so that when a magnetic force induced by a magnet (for example, a neodymium magnet) is applied to this site, the islet cells which have uptaken several GC-SPIOs settle in Mediate 1 because induction by the magnetic force is applied to the islet cells. As a result, it is possible to reduce the loss of xenogeneic islet cells that had been non-specifically generated at the time of transplantation due to existing blood pressure and blood flow velocity. Further, as the loss of islet cells is reduced, the number of islet cells transplanted may be reduced, so that the fatigue of an individual to be treated may be reduced, and an increase in the delivery rate of islet cells to the liver lobes may improve the blood glucose regulation function far more than the existing case.

The transplantation of islet cells prepared by the above method may be performed by selecting an appropriate transplant site known in the art (for example, under the renal capsule, the hepatic portal vein, and the like) and performing a known method at the transplant site, for example, a method of percutaneously injecting a prepared islet into the liver through the hepatic portal vein under ultrasound fluoroscopy without any abdominal incision. For an islet cell condition suitable for transplantation, as an ABO-matched type, when the number of islets (islet) is 5,000 kg (object body weight) or more, the islet purity is 30% or more, the final volume is 10 ml or less, and cells are Gram-negative and endotoxin negative, the islet cell condition becomes a transplantation condition (Shapiro J et al., International trial of the Edmonton protocol for islet transplantation, N Engl J Med 355:1318-1330(2006)).

As the method for preparing islet cells for transplantation of the present invention relates to the above-described composition for transplanting islet cells, overlapping contents will be omitted in order to avoid excessive complexity in the description of the present specification.

Further, the present invention provides an MRI imaging composition including biocompatible polymer-glycyrrhizin conjugate-coated magnetic nanoparticles (GC-SPIO).

According to an exemplary embodiment of the present invention, the magnetic nanoparticles may be selected from the group consisting of iron (Fe), cobalt (Co), nickel (Ni), manganese (Mn), gadolinium (Gd), oxides thereof, or alloys thereof, but are not limited thereto.

As the MRI imaging composition of the present invention relates to a method for using biocompatible polymer-glycyrrhizin conjugate-coated nanoparticles included in the above-described composition for transplanting islet cells of the present invention, overlapping contents will be omitted in order to avoid excessive complexity in the description of the present specification.

According to an exemplary embodiment of the present invention, the GC-SPIO may function as a negative contrast agent (T2 contrast agent) that darkens the corresponding site through magnetic field disturbance in an MRI device due to the inherent properties of iron oxide nanoparticles.

In addition, the present invention may provide a method for transplanting islet cells including GC-SPIO, and the transplantation may be performed in a magnetic induction environment. In this case, since the islet cells may be induced by applying a magnetic force to the transplant site, there is an advantage in that cell transplantation efficiency may be improved.

### Advantageous Effects

The present invention relates to glycyrrhizin-glycol chitosan conjugate-coated nanoparticles (GC-SPIO), islet cells, prepared using the same, for transplantation, and an MRI imagining composition comprising the same, and the GC-SPIO can bind to an HMGB1 protein to reduce the level of autoimmune response, thereby maintaining the insulin secretory function of the islet cells, and thus enabling long-term blood glucose regulation.

Further, islet cells including the biocompatible polymer-glycyrrhizin conjugate-coated magnetic nanoparticles have an advantage of being able to be tracked in real-time through MRI, and has an advantage in that the amount of transplanted islet cells can be quantitatively confirmed.

### DESCRIPTION OF DRAWINGS

FIG. 1 schematically illustrates a process of synthesizing a glycyrrhizin-glycol chitosan conjugate (GC).

FIG. 2A is a result of analyzing glycol chitosan by Fourier transform infrared spectroscopy.

FIG. 2B is a result of analyzing glycyrrhizin-glycol chitosan conjugate by Fourier transform infrared spectroscopy.

FIG. 2C is a result of analyzing iron oxide nanoparticles (bare SPIO) by Fourier transform infrared spectroscopy.

FIG. 2D is a result of analyzing glycyrrhizin-glycol chitosan conjugate-coated iron oxide nanoparticles (GC-SPIO) by Fourier transform infrared spectroscopy.

FIG. 3A is a result of measuring the potential of bare SPIO and GC-SPIO.

FIG. 3B is a result of confirming the particle size of bare SPIO and GC-SPIO by transmission electron microscopy.

FIG. 4 is a set of results of confirming whether islet cells uptake GC-SPIO, by transmission electron microscopy, after being treated with GC-SPIO by various methods: Unlabeled islet-control, GC-SPIO-untreated islet cells; Random uptake-islet cells simply treated with GC-SPIO; With Magnet-islet cells where a magnetic force is induced after GC-SPIO treatment; and On/Off system-islet cells where a cycle of magnetic force induction for 1 minute, pipetting, and culture for 1 minute without any magnetic force after GC-SPIO treatment is repeated.

FIG. 5 is a result of quantitatively analyzing the uptake of GC-SPIO after islet cells are treated with GC-SPIO by various methods (simple treatment, magnetic force induction, magnetic force induction+pipetting, on/off system): SPIO without magnet-islet cells simply treated with GC-SPIO; SPIO with magnet(On)-islet cells treated with GC-SPIO, and then brought into contact with a magnet; SPIO with magnet (On+Pipetting)-islet cells are treated with GC-SPIO, and then brought into contact with magnet+pipetting; and SPIO with magnet(On/Off)-islet cells treated with GC-SPIO, and then treated with an on/off cycle.

FIG. 6 is a result of confirming cell viability after islet cells are treated with GC-SPIO by various methods (magnetic force induction, magnetic force induction+pipetting, on/off system): intact islet-control, intact islet cells.

FIG. 7 is a result of quantitatively analyzing the uptake of GC-SPIO after islet cells are treated with GC-SPIO at various concentrations by an on/off system method.

FIG. 8 is a result of confirming cell viability after islet cells are treated with GC-SPIO at various concentrations by an on/off system method.

FIG. 9 is a set of results of quantitatively analyzing the uptake of GC-SPIO after islet cells are treated with GC-SPIO while varying the number of on/off cycles.

FIG. 10 is a set of results of confirming the change in cell viability depending on the culture period after islet cells are treated with GC-SPIO by an on/off system method.

FIG. 11 is a result of confirming the level of the HMGB1 protein released from the cell nucleus after islet cells are treated with GC-SPIO.

FIG. 12 is a result of confirming the liver lobes in which a large amount of methylene blue solution is found after the methylene blue solution is injected into mice by hepatic portal vein transplantation: Control, Right anterior (RA), Right posterior (RP), Left, Caudate 1 (C1), Caudate 2 (C2), Mediate 1 (M1), and Mediate 2 (M2).

FIG. 13 is a result of quantifying a methylene blue solution found in each hepatic lobe after the methylene blue solution is injected into mice by hepatic portal vein transplantation: Control; M1 and M2 are Mediate M1 and M2, respectively; C1 and C2 are Caudate 1 and Caudate 2, respectively; L is Left; RP is right posterior; and RA is right anterior.

FIG. 14A is a result of confirming the secretion degree of insulin in the absence of magnetic force induction after islet cells which have uptaken GC-SPIO are injected into mice by hepatic portal vein transplantation.

FIG. 14B is a result of confirming the secretion degree of insulin in the presence of magnetic force induction after islet cells which have uptaken GC-SPIO are injected into mice by hepatic portal vein transplantation.

FIG. 15 is a result of quantifying the secretion degree of insulin depending on the presence or absence of magnetic force induction after islet cells which have uptaken GC-SPIO are injected into mice by hepatic portal vein transplantation.

FIG. 16A is a result of confirming the degree of engrafting islet cells in the absence of magnetic force induction after islet cells which have uptaken GC-SPIO are injected into mice by hepatic portal vein transplantation.

FIG. 16B is a result of confirming the degree of engrafting islet cells in the presence of magnetic force induction after islet cells which have uptaken GC-SPIO are injected into mice by hepatic portal vein transplantation.

FIG. 17 is a result of quantifying the engrafting of islet cells depending on the presence or absence of magnetic force induction after islet cells which have uptaken GC-SPIO are injected into mice by hepatic portal vein transplantation.

FIG. 18 is a set of results of isolating the liver lobes and confirming the liver lobes by a microscope after islet cells which have uptaken GC-SPIO are injected into mice by hepatic portal vein transplantation.

FIG. 19 is a set of results of isolating the liver lobes and analyzing the liver lobes by MRI after islet cells which have uptaken GC-SPIO are injected into mice by hepatic portal vein transplantation.

FIG. 20 is a result of confirming the change in blood glucose depending on the presence or absence of magnetic force induction after only islet cells are injected into a diabetic animal model or islet cells which have uptaken GC-SPIO are injected into a diabetic animal model.

FIG. 21 is a schematic view schematically illustrating a method of using the GC-SPIO of the present invention and islet cells which have uptaken the same.

### MODES OF THE INVENTION

Hereinafter, the present invention will be described in more detail through Examples. These Examples are provided only for more specifically describing the present invention, and it will be obvious to a person with ordinary skill in the art to which the present invention pertains that the scope of the present invention is not limited by these Examples according to the gist of the present invention.

### Example 1: Preparation of Glycyrrhizin-Glycol Chitosan-Coated Iron Oxide Nanoparticles

### 1-1. Synthesis of Glycyrrhizin-Glycol Chitosan

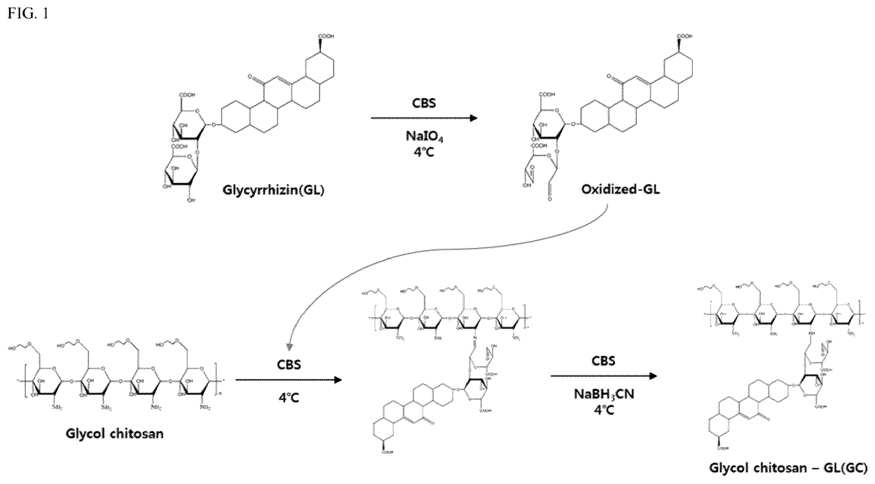

Glycyrrhizin-glycol chitosan (GC) was prepared by the synthesis method disclosed in FIG. 1. 411.47 mg of glycyrrhizic acid ammonium salt (Sigma Aldrich, USA) and 205.74 mg of glycol chitosan (WAKO PURE CHEMICAL INDUSTRIES, Japan) were added to and dissolved in 10 ml and 20 ml of a carbonate buffer with a pH of 9.5 at 4° C., respectively, and 214 mg of sodium periodate (Sigma Aldrich) was dissolved in 20 mL of tertiary distilled water (DW). When the sodium periodate was completely dissolved, the resulting solution was added to 10 ml of the glycyrrhizin solution and the resulting mixture was reacted at 4° C. for 90 minutes under a condition where light was blocked. When glycol chitosan was completely dissolved, the resulting solution was added to the solution in which glycyrrhizin and sodium periodate were dissolved, and the resulting mixture was reacted at 4° C. for about 24 hours under a condition where light was blocked. Thereafter, 15μl of cyano borohydride was added thereto, and the resulting mixture was reacted at 4° C. for 24 hours under a condition where light was blocked to stabilize the resulting product by changing the secondary amide bond into the primary amide bond. After the reaction was completed, the reaction solution was transferred to a semipermeable membrane (3,500 to 5,000 Da molecular weight cutoff; Membrane Filtration Products, USA), and then dialysis was performed in 4 L of a carbonate-bicarbonate buffer for 48 hours, and then for 72 hours using 4 L of tertiary distilled water. When the dialysis was completed, the solution was frozen with liquid nitrogen and freeze-dried for 3 days to obtain glycyrrhizin-glycol chitosan in the form of a powder (hereinafter referred to as “GC”)(FIG. 1).

### 1-2. Preparation of GC-Coated Iron Oxide Nanoparticles

After a 3-neck flask was washed with tertiary distilled water (DW), 30 ml of tertiary distilled water was put thereinto, and the flask was covered with a rubber stopper. Oxygen in the tertiary distilled water was removed by injecting a nitrogen gas into the 3-neck flask for 30 minutes, and 0.28 g of iron (II) chloride tetrahydrate and 0.56 g of iron (III) chloride hexahydrate were added to the tertiary distilled water. After 8 ml of ammonium hydroxide was added dropwise thereto, iron oxide was precipitated by stirring the resulting mixture for 30 minutes, and the precipitated iron oxide nanoparticles (superparamagnetic iron oxide nanoparticles: hereinafter, referred to as SPIO) were washed three times with tertiary distilled water in order to remove ammonium hydroxide. After 100 mg of glycyrrhizin-glycol chitosan (GC) was added to 10 ml of tertiary distilled water and the resulting mixture was stirred, this solution was mixed with the SPIO. A mixed solution of SPIO and GC was stirred at 80° C. for 2 hours, and washed three times with tertiary distilled water. Thereafter, the mixed solution was sonicated under specific conditions: 39% amplification, On time: 2 seconds, Off time: 2 seconds, total time: 1 hour. GC-uncoated SPIO was removed by ultracentrifugation (4,000 rpm, 1000 seconds, 4° C.) and finally filtered with filters having a pore size of 800 nm and 400 nm to obtain glycyrrhizin-glycol chitosan conjugate-coated superparamagnetic iron oxide nanoparticles (hereinafter, referred to as GC-SPIO).

### 1-3. GC-SPIO Analysis: Fourier Transform Infrared Spectroscopy

Whether the GC and SPIO were bound was confirmed by a Nicolet™ iS™ 50FTIR Spectrometer (Thermo Scientific, USA) as a Fourier transform infrared spectroscopy (ATR-FTIR) device.

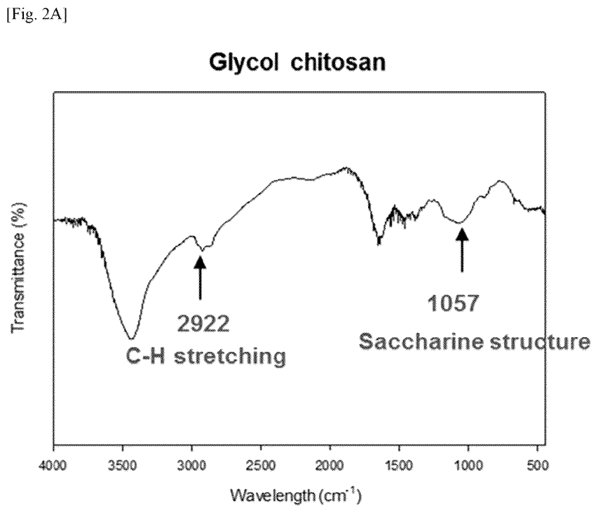

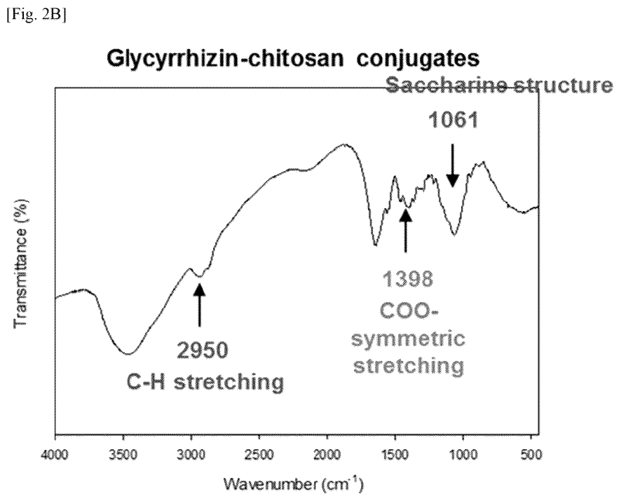

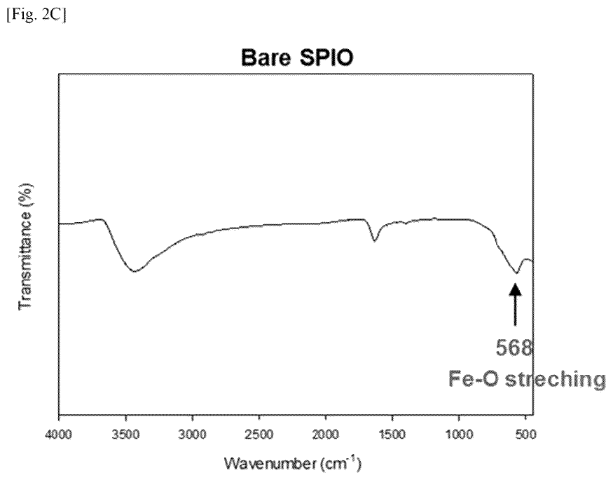

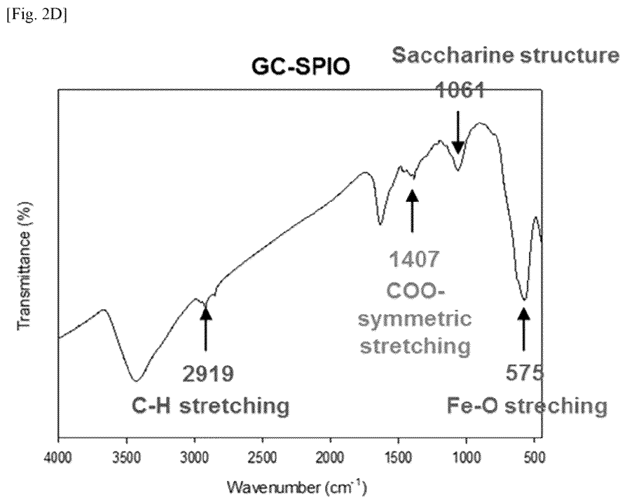

As a result, as illustrated in FIG. 2, it could be confirmed that C—H stretching present in existing glycol chitosan, saccharine structure peaks (2922 cm⁻¹, 1057 cm⁻¹), and a COO-peak (1398 cm⁻¹) were all present in the synthesized GC-SPIO. This means that glycyrrhizin and glycol chitosan were properly conjugated.

Further, as a result of measuring ATR-FTIR spectroscopy of SPIO and GC-SPIO, an Fe—O stretch peak (568 cm⁻¹) was confirmed. The C—H stretching present in GC and saccharine structure peaks (2922 cm⁻¹, 1057 cm⁻¹) were also observed in GC-SPIO which is a final synthetic material. Through this result, it could be seen that the final synthetic material GC-SPIO was properly synthesized.

### 1-4. GC-SPIO Analysis: Zeta Charge and Size

1 ml (104.5 μg/ml) of GC-SPIO was put into a disposable cuvette and the zeta potential was measured by Nano ZS (Malvern, UK).

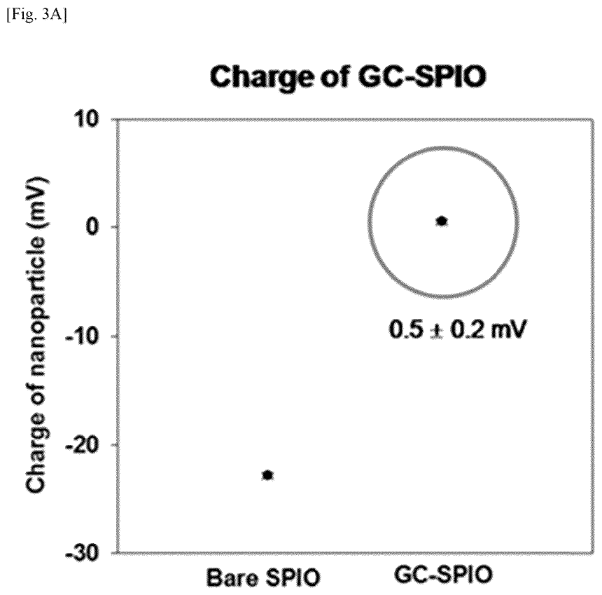

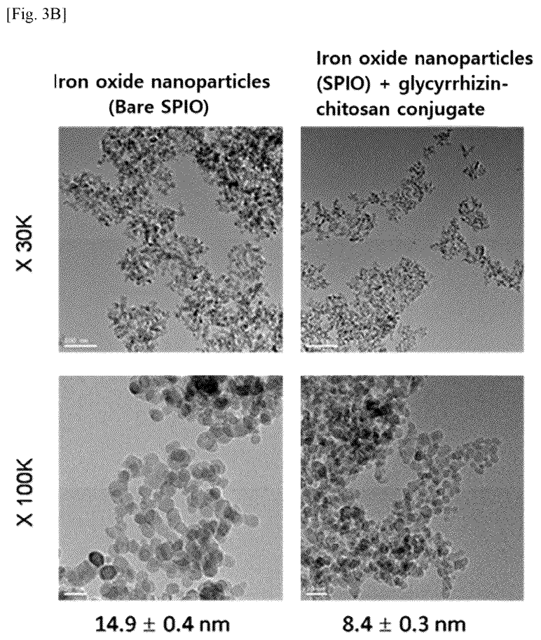

As a result of measurement, as illustrated in FIG. 3A, although GC-uncoated SPIO (hereinafter, referred to as bare SPIO) exhibited a charge of about −20 mV, it could be seen iron oxide nanoparticles were normally coated with GC by confirming that GC-SPIO exhibited a charge of about 0.5±0.2 mV due to a positively charged amine group in chitosan.

In addition, the sizes of SPIO and GC-SPIO were confirmed by transmission electron microscopy (TEM). As a result, as illustrated in FIG. 3B, it could be seen that the particle size was decreased due to the GC coating by confirming that the size of bare SPIO was about 14.9±0.4 nm, and the size of GC-SPIO was about 8.4±0.3 nm. Due to the reduced particle size, GC-SPIO may be better absorbed into the islet cells.

### Example 2: Confirmation of GC-SPIO Uptake of Islet Cells

### 2-1. Isolation of Islet Cells (Pancreatic Islets)

Collagenase P was dissolved at a concentration of 1 mg/kg in a Hanks' balanced salt solution (HBSS), and the resulting solution was intraductally injected into male SD rats. Thereafter, the pancreas was isolated and stored in water at 37° C. for 15 minutes, and the isolated islet cells were washed with Medium 199. The washed islet cells were purified, and further purified by centrifugation with Histopaque (Sigma, USA). The purified islet cells were cultured in RPMI-1640 (Invitrogen, USA) containing 10% bovine fetal serum and 1% antibiotics for 24 hours.

### 2-2. Confirmation of GC-SPIO Uptake of Islet Cells

Although GC-SPIO may be absorbed into islet cells by endocytosis, GC-SPIO has a disadvantage in that the uptake efficiency is low and the uptake randomly occurs. Accordingly, a new method for efficient uptake of GC-SPIO in islet cells was devised. Four experimental groups were a control (unlabeled islets)(GC-SPIO untreated), a random uptake group (random uptake), a magnetic force induction group (with magnet), and an on/off system treatment group (on/off system). The control is an experimental group in which islet cells were not treated with GC-SPIO, the random uptake group is an experimental group in which islet cells were simply treated with GC-SPIO, the magnetic force induction group is an experimental group in which a magnetic force was continuously generated after islet cells were treated with GC-SPIO, and the on/off system treatment group is an experimental group in which a process of GC-SPIO treatment, generation of the magnetic force, pipetting and culturing without any magnetic force was performed on islet cells.

Specifically, after the islet cells (200 IEQ) isolated in Example 2-1 were cultured in a 35π petri dish and treated with GC-SPIO at a concentration of 109.5 μg/ml, a magnetic force was generated for 1 minute, and then pipetting was performed. Thereafter, the islet cells were cultured for 1 minute without generating any magnetic force. The process of GC-SPIO treatment, generation of magnetic force, pipetting, and culturing without any magnetic force corresponds to one cycle, and such a method is named an on/off system.

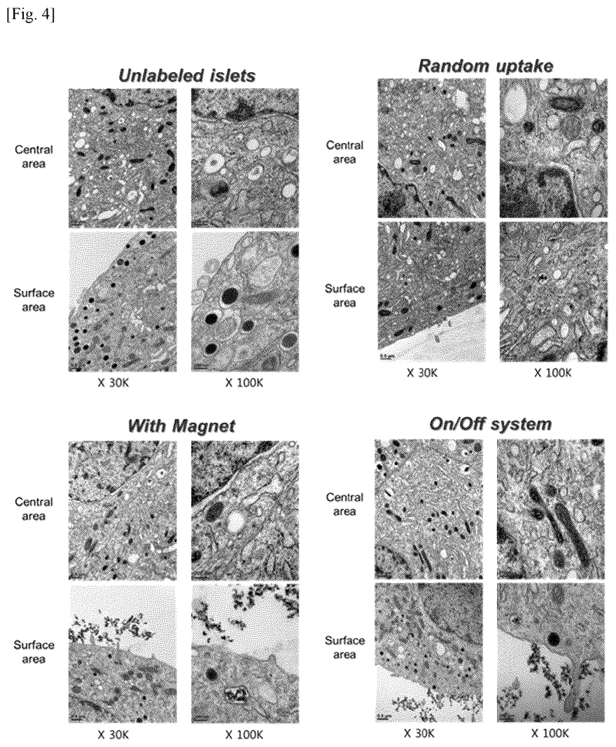

Thereafter, as a result of observing the cells by transmission electron microscopy, as illustrated in FIG. 4, it could be seen that GC-SPIO was uptaken in the islet cells in three experimental groups except for the control. In particular, it could be confirmed that the largest amount of GC-SPIO was uptaken in the on/off system treatment group.

Furthermore, the amount of GC-SPIO uptaken in the islet cells was quantitatively analyzed according to the manufacturer's protocol using an iron colorimetric assay kit (Biovision, USA). First, a diluted iron standard was made by mixing 10 μl of an iron standard included in the kit with 990 μl of tertiary distilled water (DW), and a standard curve was drawn by mixing the diluted iron standard with an assay buffer at a ratio of 0:100, 2:98, 4:96, 6:94, 8:92, and 10:90.

Next, after the islet cells (200 IEQ) were cultured in a 96-well plate, GC-SPIO (10 μM) was added thereto, and each experimental group was treated with the corresponding condition: a magnetic force non-treatment group (GC-SPIO without magnet); a magnetic force treatment group (GC-SPIO with magnet(on)); a magnetic force and pipetting treatment group (GC-SPIO with magnet (on+pipetting)); and an on/off system treatment group (GC-SPIO with magnet (on/off)). Thereafter, 5 ml of an iron reducing agent was added to each well and mixed for 30 minutes, and 100 ml of an iron probe was additionally aliquoted into each well in order to express fluorescence. The wells were wrapped with foil in order to block light, and then shaken at room temperature for 1 hour. The amount of GC-SPIO uptaken by the islet cells was confirmed by measuring the absorbance at a wavelength of 593 nm in a dark place.

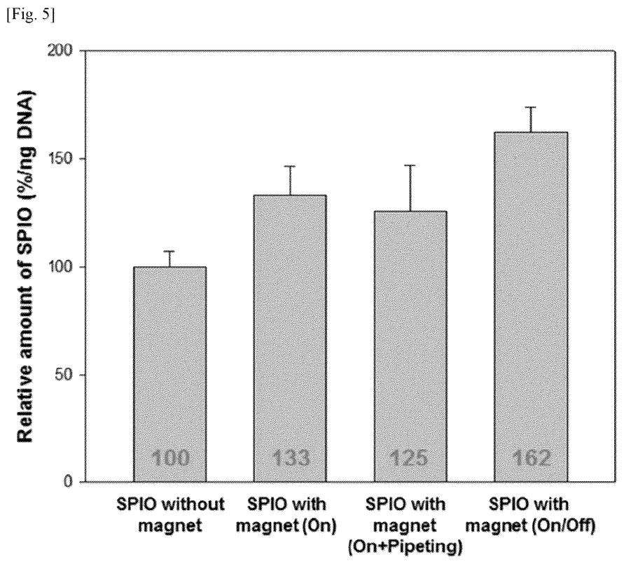

As a result, as illustrated in FIG. 5, it could be confirmed that the largest amount of GC-SPIO was introduced into the islet cells in the on/off system treatment group.

### 2-3. Confirmation of Change in Viability of Islet Cells by On/Off System

In order to confirm whether the GC-SPIO uptake of islet cells induced cytotoxicity, islet cells (200 IEQ) were cultured in a 24-well plate, and then treated with GC-SPIO (10 μM) for 30 minutes, and each experimental group was additionally treated with the corresponding condition: a control (intact islet)(GC-SPIO untreated); a magnetic force non-treatment group (GC-SPIO without magnet); a magnetic force treatment group GC-SPIO with magnet(on)); and an on/off system treatment group (GC-SPIO with magnet(on/off)). Thereafter, the cells were cultured at 37° C. for 4 hours by adding a CCK solution corresponding to 10% of the culture medium to each well, the cells were recovered, and then cell viability was measured by measuring the absorbance at a wavelength of 450 nm in a dark place.

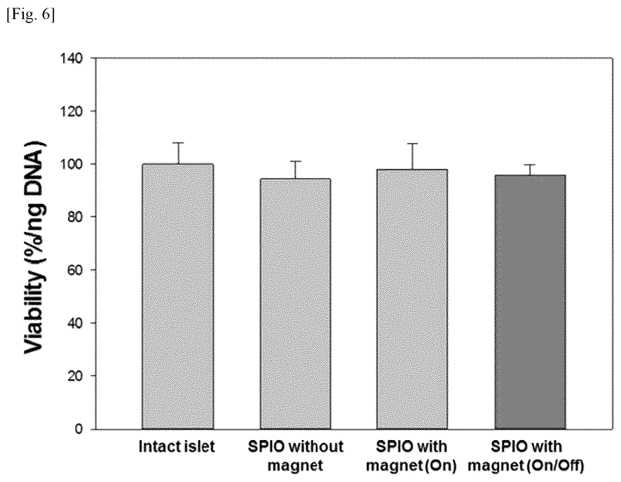

As a result of measurement, as illustrated in FIG. 6, it could be confirmed that a there was no significant difference in cell viability between experimental groups. Therefore, it could be seen that when GC-SPIO is uptaken into islet cells, the on/off system, which is the most effective method, has no cytotoxicity.

### Example 3: Confirmation of Optimal Conditions of Magnetic Force On/Off System

### 3-1. Optimization of GC-SPIO Treatment Concentration

After islet cells (200 IEQ) were cultured in a 96-well plate, the islet cells were treated with various concentrations of GC-SPIO (0, 2.5, 5, 10, 20, and 45 μg/ml) by an on/off system method. Specifically, a process of treating a 35π petri dish including islet cells and 3 ml of an RPMI (PBS 10%, PS 1%) medium with GC-SPIO at the corresponding concentration, applying a magnetic force thereto for 1 minute, performing pipetting for 1 minute, and then culturing the islet cells without any treatment with a magnetic force for 1 minute was performed 12 times (1 cycle×12) in total. Thereafter, GC-SPIO which was not uptaken in the islet cells was separated from the islet cells by a cell strainer. Next, an iron absorbance analysis kit was used for analysis according to the method of Example 2-2.

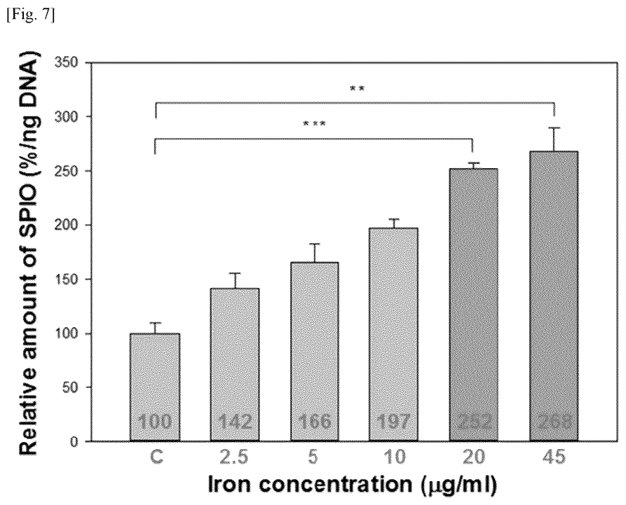

As a result, as illustrated in FIG. 7, it could be seen that the amount uptaken into the islet cells was increased according to the treatment concentration of GC-SPIO. Based on the control (C), in the case of treatment concentrations of 2.5 μg/ml, 5 μg/ml, 10 μg/ml, 20 μg/ml, and 45 μg/ml, values such as 142, 166, 197, 252, and 268 were exhibited, respectively, so that it was confirmed that a significant difference was exhibited between the control and each experimental group. However, statistical significance was not exhibited between the GC-SPIO treatment concentration 20 μg/ml group (252) result and the GC-SPIO treatment concentration 45 μg/ml group (268) result. Through this, it could be seen that a positive correlation was present between the treatment concentration and the GC-SPIO uptake amount until the GC-SPIO treatment concentration 20 μg/ml, but the correlation with the uptake amount was weak at the treatment concentration exceeding 20 μg/ml.

### 3-2. Confirmation of Islet Cell Viability

After the GC-SPIO was uptaken into islet cells under the same conditions as in 3-1, a CCK analysis was performed by isolating the cells.

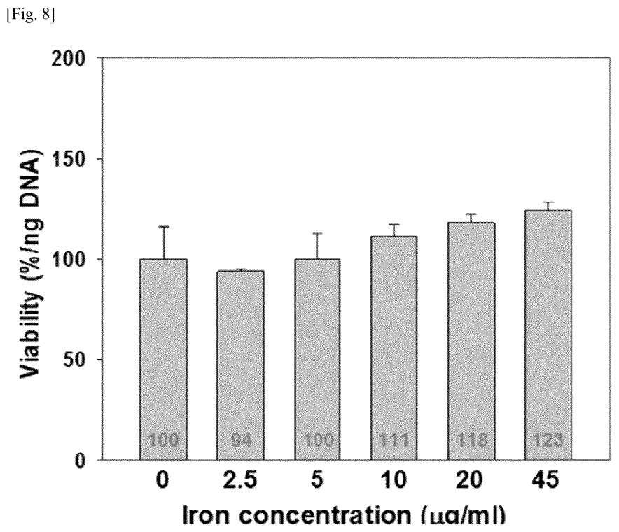

As a result, as illustrated in FIG. 8, a significant difference in cell viability could not be confirmed between the control and each experimental group, and it could be concluded that the GC-SPIO treatment concentration was irrelevant to the toxicity of islet cells.

In consideration of the fact that there was no difference in the uptake amount of GC-SPIO at 20 μg/ml and 45 μg/ml by summarizing the results in Example 3-1 and the filtering process at a treatment concentration of 45 μg/ml takes a long time together, the optimum concentration of GC-SPIO was set to be 20 μg/ml.

### 3-3. Optimization of Magnetic Force On/Off System Cycle Number

When GC-SPIO was uptaken into islet cells using the magnetic force on/off system, the uptake amount of GC-SPIO according to the on/off cycle number was confirmed.

Specifically, a 35π petri dish including the islet cells and 3 ml of an RPMI (PBS 10%, PS 1%) medium was treated with GC-SPIO (20 μg/ml), and the on/off cycle was performed 0 time (control), 4 times, 8 times, and 12 times for each experimental group. Thereafter, GC-SPIO which was not uptaken in the islet cells was separated from the islet cells by a cell strainer. Next, an iron absorbance analysis kit was used for analysis according to the method of Example 2-2.

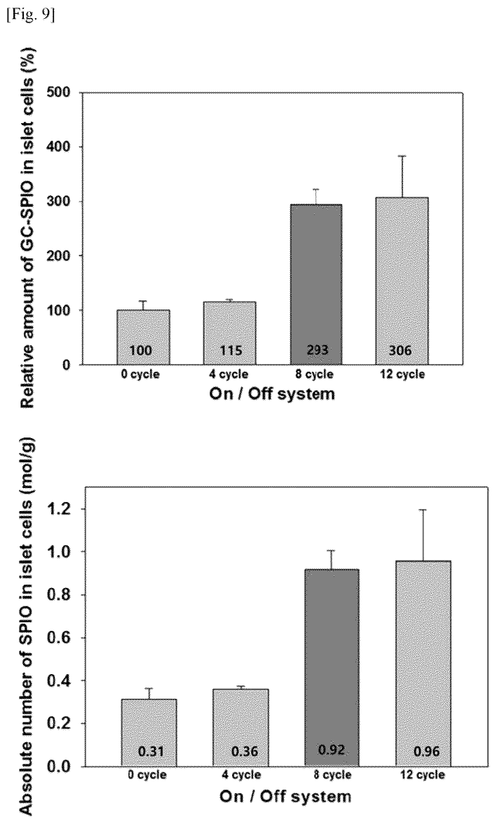

As a result of analysis, as illustrated in FIG. 9, based on the control, it could be seen that when the on/off cycle was performed 4 times, there was no significant difference in the uptake amount of GC-SPIO, and when the on/off cycle was performed 8 times, the uptake amount of GC-SPIO was increased about 2.9-fold. In contrast, it could be confirmed that when the on/off cycle was performed 12 times, the uptake amount of GC-SPIO was increased about 3-fold, which was similar to that when the on/off cycle was performed 8 times.

Therefore, in consideration of the efficiency of the experiment, it could be concluded that performing the on/off cycle 8 times is the most optimal cycle number.

### 3-4. Confirmation of Change in Islet Cell Viability in Optimal On/Off System

In order for the transplanted islet cells to maintain the blood glucose regulation function for a long period, the viability of the islet cells needs to be maintained for a long period of time. Therefore, the viability of islet cells which have uptaken GC-SPIO in the optimal on/off system confirmed in the present invention was confirmed.

Specifically, a 35π petri dish including the islet cells and 3 ml of an RPMI (PBS 10%, PS 1%) medium was treated with GC-SPIO (10 or 20 μg/ml), the on/off cycle was performed 8 times, and then the islet cells were cultured for 1, 3, 5, and 7 days. When the culture was completed, cell viability was confirmed by a CCK analysis.

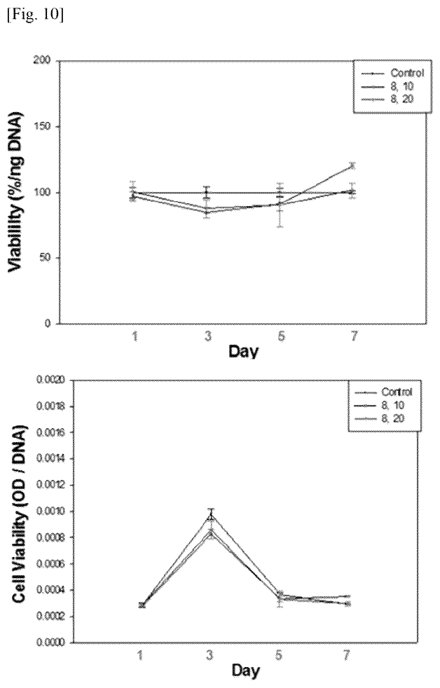

As a result, as illustrated in FIG. 10, a significant difference in cell viability between the control (intact islet cells) and each experimental group could not be confirmed. This result means that the optimal on/off system of the present invention does not induce cytotoxicity, and thus, the islet cells can settle in the liver tissue after transplantation and survive sufficiently until the insulin secretory function is fulfilled.

### Example 4: Confirmation of Effects of Islet Cells Which Have Uptaken GC-SPIO

### 4-1. GC-SPIO Inhibitory Effect on HMGB1 Protein Release

The amount of HMGB1 protein released from the islet cells was confirmed by an HMGB1 Elisa kit (ELABSCIENCE, USA). A total of 6 experimental groups were designed by dividing the experimental group into a streptozotocin treatment group and a non-treatment group and again classifying each experimental group into a control (Control), an SPIO treatment group (Bare-SPIO), and a GC-SPIO treatment group (GC-SPIO).

First, 200 islet equivalent (IEQ) islet cells were aliquoted into each well of a 96-well cell culture plate and cultured, and CC-SPIO was uptaken into the islet cells under an uptake optimal condition of 20 μg/ml treatment with 8 cycles of the on/off system. Thereafter, 1.5 mM streptozotocin (Stz) was added to induce the release of HMGB1 protein from islet cells, and the cells were cultured for 2 hours. Next, 100 μl of a color solution included in the kit was added to each well, and the mixture was reacted while being shaken at room temperature for 30 minutes. Finally, 100 ml of a stop solution was aliquoted into each well and gently shaken, and absorbance was measured at a wavelength of 450 mm in the dark within 1 hour.

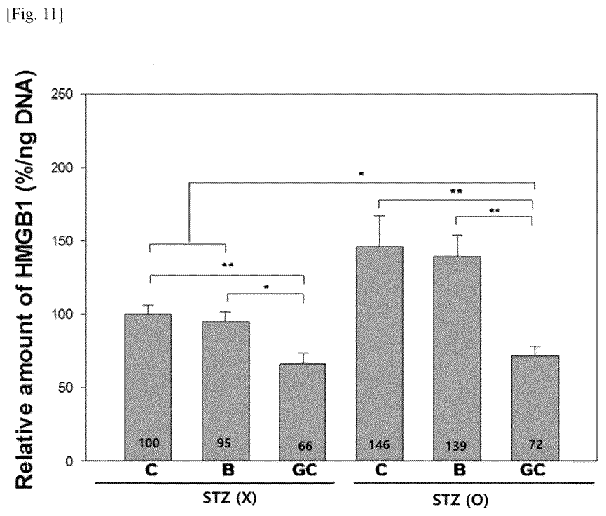

As a result, as illustrated in FIG. 11, it was confirmed that there was no significant difference in amount of HMGB1 protein released between the control (C) and the SPIO treatment group (B), but the released HMGB1 protein in the GC-SPIO treatment group was remarkably decreased compared to that in the control, and this tendency was exhibited even when cell stress was induced by streptozotocin. This result means that GC-SPIO can effectively inhibit the release of the HMGB1 protein.

### 4-2. Confirmation of Liver Lobes to Which Largest Amount of Transplanted Material is Delivered

An experiment was performed as follows to confirm the liver lobes to which the largest amount of islet cells was delivered during islet cell transplantation. After zoletil and rumpun were injected into the abdominal cavity of balb/c mice at a ratio of 9:1 to anesthetize them, a methylene blue solution was injected by hepatic portal vein transplantation. After waiting for about 24 hours for tissue fixation of the methylene blue solution, all liver lobes were isolated by sacrificing the mice. The isolated liver lobes were fixed with a 10% formalin solution, made into a paraffin block, and cut into a thickness of 5 μm to prepare a tissue section slide. Thereafter, paraffin was removed from the tissue section slide, the slide was rehydrated, and then the tissue section was stained with a Harris hematoxylin solution and an eosin solution, and observed under an optical microscope.

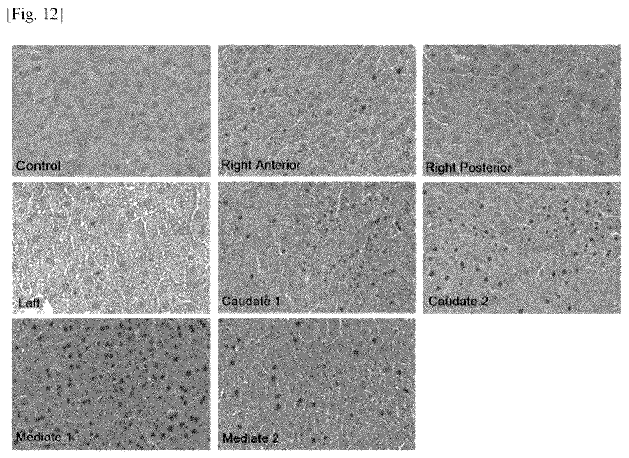

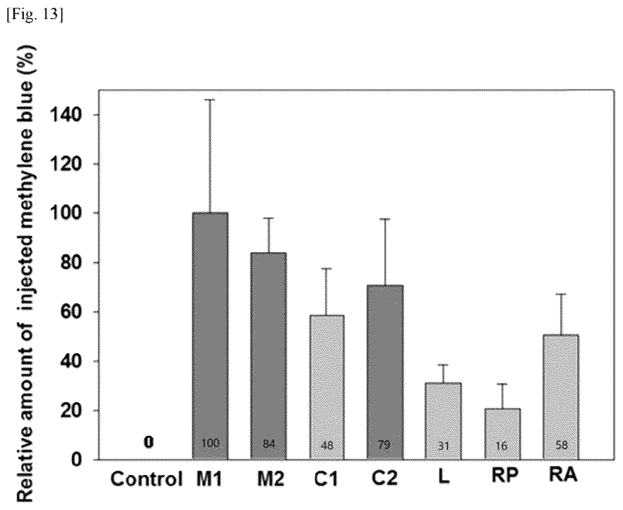

As a result, as illustrated in FIG. 12, it could be confirmed that the methylene blue solution was more distributed in the mediate liver lobes than in the other liver lobes: caudate, right anterior, right posterior. In addition, as a result of quantitative analysis by image J, as illustrated in FIG. 13, it could be seen that the largest amount of methylene blue solution was distributed in the mediate liver lobes, and the methylene blue solution was present in the largest amount in Mediate 1 among the mediate liver lobes. Through this result, it can be seen that the generation of magnetic force centered on the mediate liver lobes is effective for increasing the success rate of islet cell transplantation.

### 4-3. Confirmation of Insulin Signals

GC-SPIO was uptaken into islet cells (700 IEQ) under optimal conditions (8 cycles, a GC-SPIO concentration of 20 μg/ml), the islet cells were injected into balb/c mice (n=4/experimental group), and then a magnetic force was generated by bringing a magnet into contact with the mediate liver lobe sites of the livers of the mice from the outside. After 24 hours, all liver lobes were removed by sacrificing the mice, and liver lobe section slides were prepared according to Example 4-2, and then paraffin was removed therefrom. Thereafter, pancreatic tissue slides among the above liver lobe section slides were reacted with an insulin antibody (1:200 dilution, mouse monoclonal antibody; Abcam, USA) and 20% goat serum at 4° C. overnight. The next day, the pancreatic tissue slides were reacted with a secondary antibody, AlexaFluor 574 goat anti-mouse antibody (1:1000 dilution; Invitrogen, USA) at room temperature in a light-blocked state for 1 hour. Thereafter, the pancreatic tissue slide was washed with PBS, stained with DAPI, and observed under a fluorescence microscope.

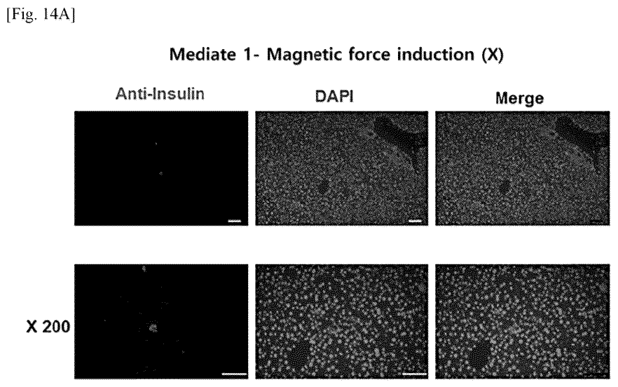

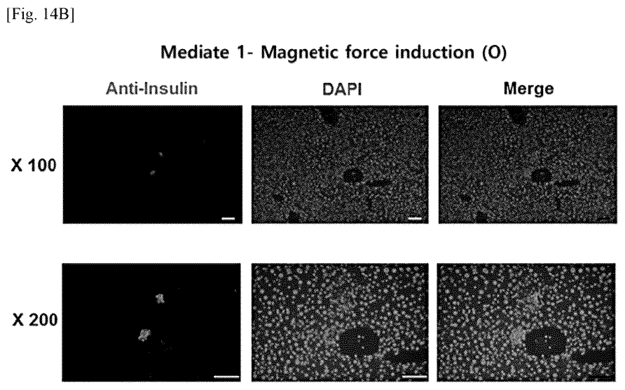

As a result, as illustrated in FIG. 14, it could be confirmed that after islet cell transplantation, in the experimental group (n=5) in which a neodymium magnet was attached to Mediate 1 to give the magnetic force induction effect, a much higher amount of insulin signal was observed than that in the experimental group (n=5) in which a neodymium magnet was not attached to Mediate 1 to give the magnetic force induction effect. From the result, it could be seen that a larger amount of islet cells which have uptaken GC-SPIO were induced in the mediate liver lobes through the magnetic force induction effect.

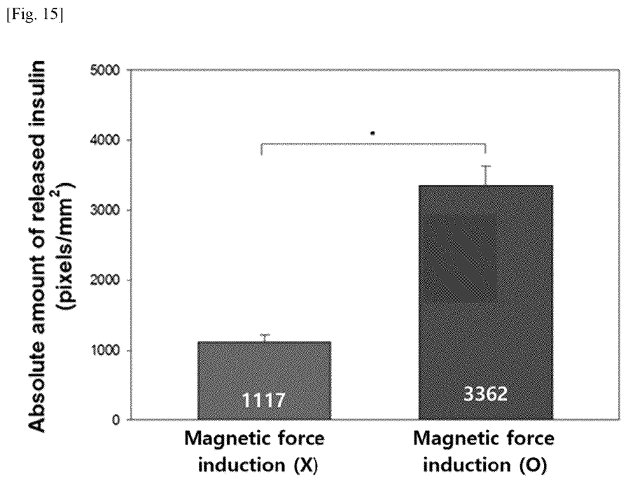

Further, as a result of quantitative analysis by image J, as illustrated in FIG. 15, it could be confirmed that the insulin signal was remarkably higher in the experimental group (magnetic induction O) to which the magnetic induction effect was given than in the experimental group (magnetic induction X) to which the magnetic induction effect was not given. As a result of taking the average of each experimental group, it was found that the insulin signal of the experimental group to which the magnetic induction effect was given was shown to be 3362 pixels/mm² and that of the experimental group to which the magnetic induction effect was not given was shown to be 1117 pixels/mm².

### 4-4. Staining With Prussian Blue

In order to confirm whether the insulin signal confirmed in Example 4-3 was due to its own islet cells originally possessed by the balb/c mice, or a signal which was induced by the magnetic force and produced from the islet cells settled in Mediate 1, staining with Prussian blue was performed.

GC-SPIO was uptaken into islet cells (700 IEQ) under optimal conditions (8 cycles, a GC-SPIO concentration of 20 μg/ml), the islet cells were injected into balb/c mice (n=3/experimental group) through the hepatic portal vein, and then a magnetic force was generated by bringing a magnet into contact with the mediate liver lobe sites of the livers of the mice from the outside. After 24 hours, all liver lobes were removed by sacrificing the mice, and liver lobe section slides were prepared according to Example 4-2, and then paraffin was removed therefrom. Thereafter, the liver lobe slide was stained twice with a staining solution (4% potassium ferrocyanide+4% hydrochloric acid) for 10 minutes each. Thereafter, the liver lobe slide was washed twice with tertiary distilled water for 3 minutes, immersed in a nuclear fast red solution, and then covered with parafilm. After the slide was rinsed with running water for 1 minute, moisture was removed and a polymounting solution was dropped thereonto, and then the slide was covered with a cover slip and observed under an optical microscope.

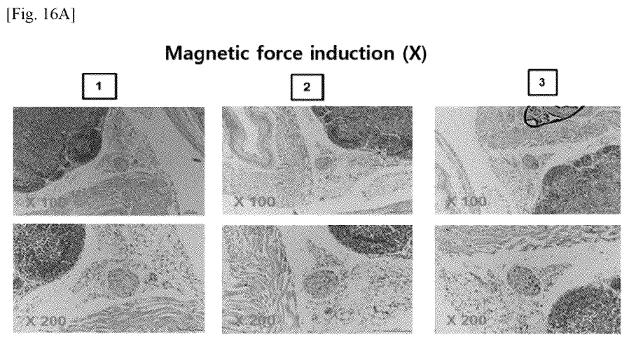

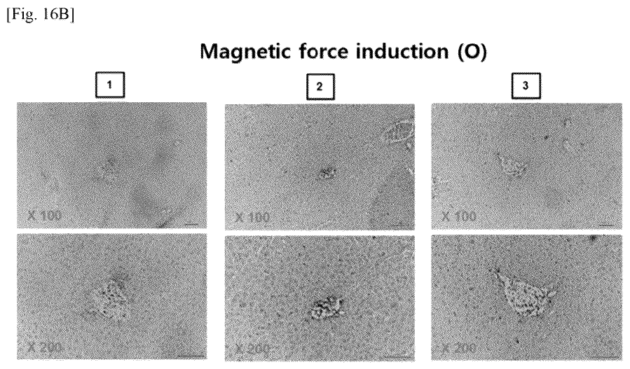

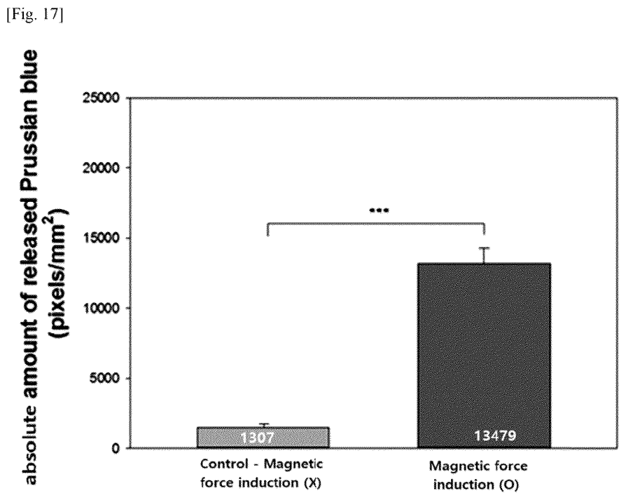

As a result of observation, as illustrated in FIGS. 16 and 17, it could be seen that the islet cells including GC-SPIO effectively settled in the mediate liver lobes of the mouse liver by confirming that the experimental group (magnetic force induction O) was clearly stained compared to the control (magnetic force induction X).

### 4-5. Magnetic Resonance Imaging (MRI) Imaging

GC-SPIO (a concentration of GC-SPIO: 20 μg/ml) was uptaken into islet cells by 8 cycles of the on-off system, and the islet cells were injected into balb/c mice, and then a magnetic force was induced. After 24 hours, all liver lobes were isolated and fixed, placed in a Petri dish, and mixed with 1% agarose gel to remove noise due to oxygen. Thereafter, a T2 MRI liver scan was performed by a 3T Skyra MRI device.

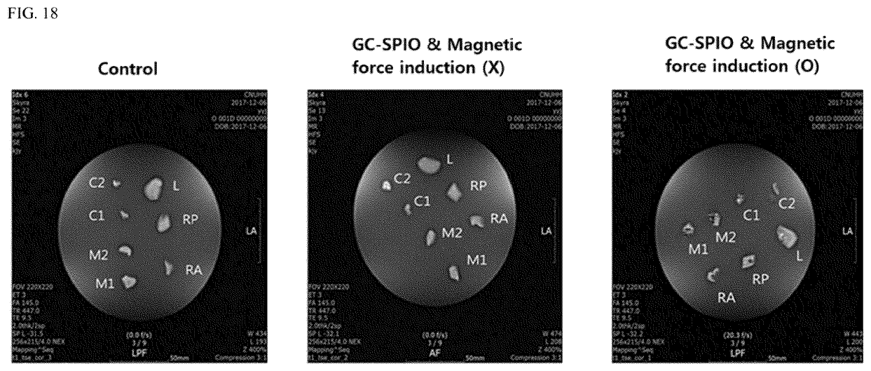

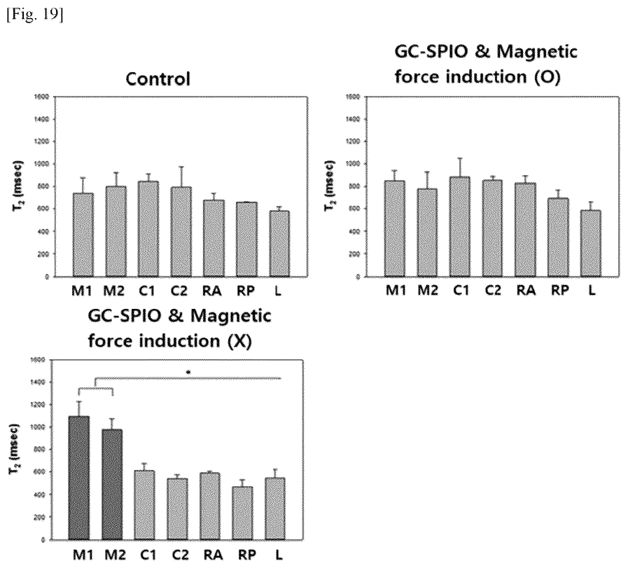

As a result, as illustrated in FIG. 18, it could be confirmed that the mediate liver lobes (M1 and M2) appeared darker in the experimental group (GC-SPIO & magnetic induction O) in which a magnetic force was induced compared to the control (GC-SPIO not injected). In addition, as a result of quantitative analysis by image J, as illustrated in FIG. 19, it could be seen that the T2 (negative contrast) signal appeared strong in the mediate liver lobes (M1 and M2) of the magnetic force-induced experimental group (GC-SPIO & magnetic force induction O). This means that a large amount of GC-SPIO was induced in the mediate liver lobes of the liver and the islet cells which have uptaken GC-SPIO were present in the mediate liver lobes of the liver. Meanwhile, through the present result, it can be seen that GC-SPIO can also be used as a T2 MRI contrast agent.

### 4-6. Effect of Islet Cells Which Have Uptaken GC-SPIO on Treatment of Diabetes

GC-SPIO (a concentration of GC-SPIO: 20 μg/ml) was uptaken into islet cells by 8 cycles of the on-off system, and the islet cells were injected into balb/c diabetic model mice (700 IEQ/mouse), and then a magnetic force was induced. Blood glucose was measured in the diabetic model mice for 2 weeks after islet cell transplantation.

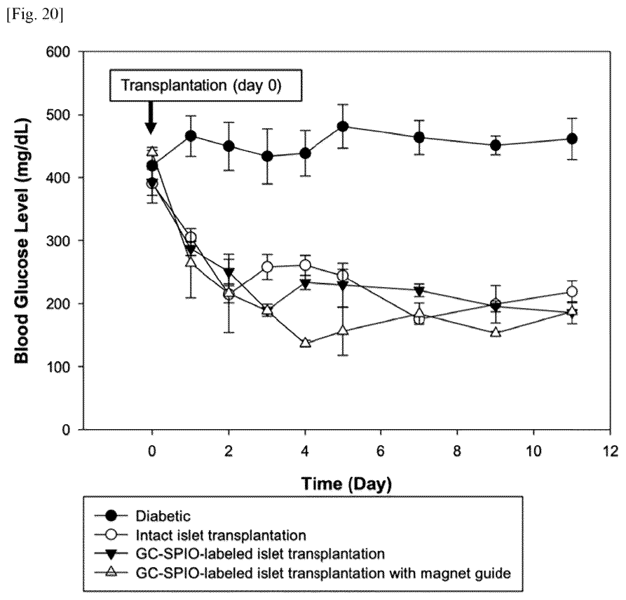

As a result, as illustrated in FIG. 20, it could be confirmed that the levels of blood glucose of the diabetic model mice were restored to normal levels in the experimental group (intact islet transplantation) in which only islet cells were transplanted, the experimental group (GC-SPIO-labeled islet transplantation) in which islet cells which have uptaken GC-SPIO were transplanted, and the experimental group (GC-SPIO-labeled islet transplantation with magnet guide) of transplantation of islet cells which have uptaken GC-SPIO+magnetic force induction, compared to the experimental group (diabetic) in which islet cells were not transplanted as the control. In particular, it could be seen that the blood glucose level of the experimental group in which islet cells were transplanted into the mediate liver lobes of the liver was also decreased to the normal level due to the magnetic force induction. This result means that even when islet cells which have uptaken GC-SPIO were transplanted into a target site due to the magnetic force, the diabetes treatment effect was maintained, and in addition to the diabetes treatment effect, islet cells which have uptaken GC-SPIO were easily removed from a target site (mediate liver lobes of the liver) by the partial resection of liver.

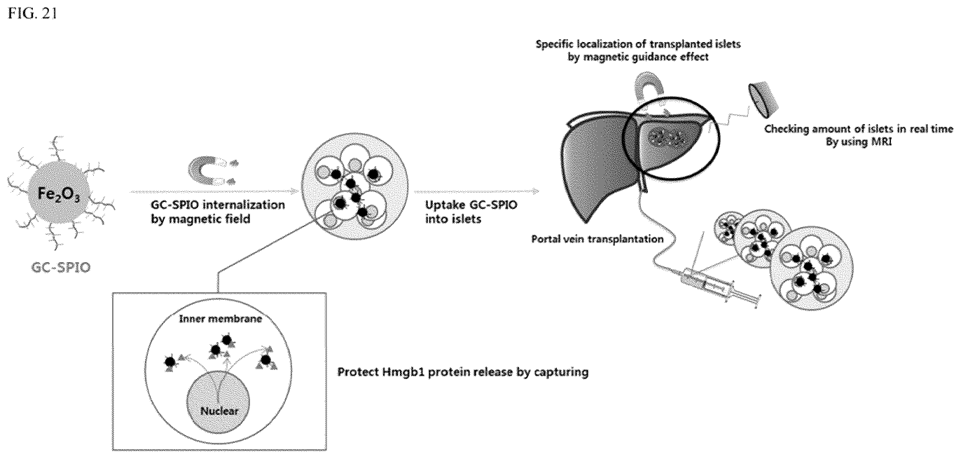

Through the results of Examples 1 to 4, it can be seen that the GC-SPIO of the present invention can be effectively uptaken into the islet cells by magnetic force induction, and the islet cells which have uptaken GC-SPIO can move to a desired target site through the magnetic force induction. Furthermore, it can be seen that the islet cells which have uptaken GC-SPIO moved to the target site stably secrete insulin, and thus can be used for the treatment of diabetes (FIG. 21).

1. A method for preparing islet cells for transplantation, the method comprising:
(a) bringing a composition for transplanting islet cells into contact with islet cells isolated from a donor;
(b) applying a magnetic force to the islet cells for 0.1 to 5 minutes; and
(c) mixing the resulting product of (b) for 0.1 to 5 minutes,
wherein the composition comprises nanoparticles coated with glycol chitosan-glycyrrhizin conjugate, and the glycol chitosan-glycyrrhizin conjugate is linked by a covalent bond.

2. The method of claim 1, wherein the glycyrrhizin is an oxidized form.

3. The method of claim 1, wherein the covalent bond is selected from the group consisting of an amide bond, a carbonyl bond, an ester bond, a thioester bond, and a sulfonamide bond.

4. The method of claim 1, wherein the nanoparticles are inorganic nanoparticles.

5. The method of claim 4, wherein the inorganic nanoparticles are magnetic nanoparticles selected from the group consisting of iron (Fe), cobalt (Co), nickel (Ni), manganese (Mn), gadolinium (Gd), oxides thereof, or alloys thereof.

6. The method of claim 1, further comprising (d) allowing the islet cells to stand for 0.1 minute to 5 minutes after Step (c).

7. The method of claim 6, wherein Steps (b) to (d) are repeated 1 time to 20 times.

8. A method for alleviating or treating an islet cell-deficiency disease in a subject in need thereof, comprising administering the islet cells for transplantation prepared by the method of claim 1 to the subject, wherein the administration is performed by an injection into hepatic portal vein of the subject.

9. The method of claim 8, wherein the islet cell-deficiency disease is a disease selected from the group consisting of type 1 diabetes, type 2 diabetes, and a diabetic chronic kidney disease.
